SEQUENCE LISTING (4 sequences)

SEQ ID NO: 1 - protein, 591 aa, Saccharomyces cerevisiae
MLKYKPLLKISKNCEAAILRASKTRLNTIRAYGSTVPKSKSFEQDSRKRTQSWTALRVGAILAATSSVAYLNWHNGQIDNEPKLDMNKQKISPAEVAKHNKPDDCWVVINGYVYDLTRFLPNHPGGQDVIKFNAGKDVTAIFEPLHAPNVIDKYIAPEKKLGPLQGSMPPELVCPPYAPGETKEDIARKEQLKSLLPPLDNIINLYDFEYLASQTLTKQAWAYYSSGANDEVTHRENHNAYHRIFFKPKILVDVRKVDISTDMLGSHVDVPFYVSATALCKLGNPLEGEKDVARGCGQGVTKVPQMISTLASCSPEEIIEAAPSDKQIQWYQLYVNSDRKITDDLVKNVEKLGVKALFVTVDAPSLGQREKDMKLKFSNTKAGPKAMKKTNVEESQGASRALSKFIDPSLTWKDIEELKKKTKLPIVIKGVQRTEDVIKAAEIGVSGVVLSNHGGRQLDFSRAPIEVLAETMPILEQRNLKDKLEVFVDGGVRRGTDVLKALCLGAKGVGLGRPFLYANSCYGRNGVEKAIEILRDEIEMSMRLLGVTSIAELKPDLLDLSTLKARTVGVPNDVLYNEVYEGPTLTEFEDA

SEQ ID NO: 2 - DNA, 1776 bp, Saccharomyces cerevisiae
atgttgaagtacaagccattgctaaagatttccaagaactgtgaagctgccatcttgagagcttccaagaccagattgaacactatcagagcttacggttctaccgttccaaagtccaagtctttcgaacaagactccagaaagcgtacacaatcttggactgccttgagagtcggtgctattctagctgccacctcttccgttgcttacttgaactggcacaatggtcaaatcgacaacgaaccaaagttggatatgaacaagcaaaagatttctccagctgaagttgccaagcacaacaaaccagacgattgttgggtcgttatcaacggttacgtctacgacttaaccagatttctacccaatcatccaggtggccaagacgttatcaagttcaacgctggcaaggatgtcactgctatcttcgaaccattgcacgctccaaacgtcatcgacaagtacattgctccagaaaagaaattgggtccattgcaaggttctatgcctccagaattggtctgtcctccatacgctccaggtgaaaccaaggaagacattgccagaaaggaacaactaaagtccttgttaccacctttggacaacattatcaacttgtacgatttcgaatacttggcttctcaaactttgaccaagcaagcctgggcttactacagctctggtgccaacgacgaagtcactcacagagaaaaccacaatgcttaccacagaatctttttcaagccaaagattttggttgacgtcagaaaggttgacatctctaccgatatgttgggttctcacgttgacgtgccattttacgtctctgctactgccttgtgcaagttgggaaatccattggaaggtgagaaggacgttgccagaggttgtggccaaggtgtcaccaaggttccacaaatgatctccactttagcttcatgttctccagaggaaattatcgaagctgctccatccgacaagcaaatccaatggtaccaattgtacgtcaactccgacagaaagattaccgacgatttggtcaagaacgtcgaaaagttgggtgtcaaggccttatttgttactgtcgatgctccaagcctaggtcaaagagaaaaggacatgaagttgaagttttccaacacaaaggctggtcccaaggctatgaagaaaaccaatgttgaagagtctcaaggtgcctccagagctttgtccaagttcatcgacccatctttaacttggaaggatatcgaagagttgaagaaaaagaccaagctaccaattgttatcaagggtgttcaaagaaccgaagacgttatcaaggctgccgaaatcggtgtctctggtgttgtcctatccaaccacggtggcagacaattggatttttcccgtgctccaatcgaagtcttggctgaaaccatgccaatcttggaacaaagaaacttgaaggacaagttggaagtttttgtcgatggtggcgtcagacgtggtacagacgtcttgaaggctctatgtttgggtgccaagggtgttggcttaggtagaccattcttgtacgccaactcctgttacggcagaaacggtgtcgaaaaggctatcgaaatcttgagagacgaaattgaaatgtctatgcgtctattgggtgttacttccatcgctgaattgaagccagatttgctagacttatctactttgaaggccagaaccgttggtgtacccaacgacgtcttgtacaacgaagtttacgaaggtcctactttgaccgaatttgaggatgcttaa

SEQ ID NO: 3 - DNA, 779 bp, Artificial sequence
  Synthetic construct
aaagtatgtcccaacaaaacctgaacgaaaccactcagaagaaggaagacaaagggaacgtcaacctgaagggacagagtttaaccaacaccggtgggggctgctgttgaacaagcgcgcctctaccttgcagacccatataatataataactaaataagtaaataagacacacgcgagaacatatatacacaattacagtaacaataacaagaggacaaatactaccaaaatgtgtggggaagcgggtaagctgccacagcaattaatgcacaacatttaacctacattcttccttatcggatcctcaaaacccttaaaaacatatgcctcaccctaacatattttccaattaaccctcaatatttctctgtcacccggcctctattttccattttcttctttacccgccaggcgtttttttctttcaaatttttttcttccttcttctttttcttccacgtcctcttgcataaataaataaaccgttttgaaaccaaactcgcctctctctctcctttttgaaatatttttgggtttgtttgatcctttccttcccaatctctcttgtttaatatatattcatttatatcacgctctctttttatcttcctttttctcctctctcttgtattcttccttcccctttctactcaaaccaagaagaaaaagaaaaggtcaatctttgttaaagaataggatcttctactacatcagcttttagatttttcacgcttactgcttttttcttcccaagatcgaaaatttactgaattaaca

SEQ ID NO: 4 - DNA, 303 bp, Artificial sequence
  Synthetic construct
gttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttg

(19) United States
(12) Patent Application Publication
(10) Pub. No.: US 2025/0043236 A1
(43) Pub. Date: Feb. 6, 2025

(54) OVER-EXPRESSION OF CYTOCHROME B2 IN YEAST FOR INCREASED ETHANOL PRODUCTION

(71) Applicant: DANISCO US INC, Palo Alto, CA (US)

(72) Inventors: Daniel Joseph MACOOL, RUTLEDGE, PA (US); Quinn Qun ZHU, WEST CHESTER, PA (US)

(21) Appl. No.: 17/439,127

(22) PCT Filed: Mar. 13, 2020
(86) PCT No.: PCT/US20/22777

§ 371 Date: Sep. 14, 2021

Related U.S. Application Data
(60) Provisional application No. 62/818,448, filed on Mar. 14, 2019.

(51) Int. Cl. C12N 1/18; C12N 9/04; C12N 15/52; C12P 7/06

(52) U.S. Cl. CPC C12N 1/185; C12N 9/0006; C12N 15/52; C12P 7/06; C12Y 101/02003

(57) ABSTRACT

Described are compositions and methods relating to modified yeast that over-express cytochrome B2. The yeast produces an increased amount of alcohol compared to parental cells. Such yeast is particularly useful for large-scale ethanol production from starch substrates.

This application claims priority to U.S. Provisional Patent Application No. 62/818,448 filed Mar. 14, 2019, the disclosure of which is incorporated by reference in its entirety.

### TECHNICAL FIELD

The present compositions and methods relate to modified yeast that over-expresses cytochrome B2. The yeast produces an increased amount of ethanol compared to their parental cells. Such yeast is particularly useful for large-scale ethanol production from starch substrates.

### BACKGROUND

First-generation yeast-based ethanol production converts sugars into fuel ethanol. The annual fuel ethanol production by yeast is about 90 billion liters worldwide (Gombert, A. K. and van Maris. A. J. (2015) Curr. Opin. Biotechnol. 33:81-86). It is estimated that about 70% of the cost of ethanol production is the feedstock.

Butanol is an important industrial chemical and drop-in fuel component with a variety of applications including use as a renewable fuel additive, a feedstock chemical in the plastics industry, and a food-grade extractant in the food and flavor industry. Accordingly, there is a high demand for alcohols such as butanol and isobutanol, as well as for efficient and environmentally-friendly production methods.

In view of the large amount of alcohol produced in the world, even a minor increase in the efficiency of a fermenting organism can result in a tremendous increase in the amount of available alcohol. Accordingly, the need exists for organisms that are more efficient at producing alcohol.

### SUMMARY

The present compositions and methods relate to modified yeast that over-express cytochrome B2. Aspects and embodiments of the compositions and methods are described in the following, independently-numbered, paragraphs.

1. In one aspect, modified yeast cells derived from parental yeast cells are provided, the modified cells comprising a genetic alteration that causes the modified cells to produce an increased amount of CYB2P polypeptides compared to the parental cells, wherein the modified cells produce during fermentation more ethanol compared to the amount of ethanol produced by otherwise identical parent yeast cells.

2. In some embodiments of the modified cells of paragraph 1, the genetic alteration comprises the introduction into the parental cells of a nucleic acid capable of directing the expression of a CYB2P polypeptide to a level above that of the parental cell grown under equivalent conditions.

3. In some embodiments of the modified cells of paragraph 1, the genetic alteration comprises the introduction of an expression cassette for expressing a CYB2P polypeptide.

4. In some embodiments of the modified cells of any of paragraphs 1-3, the amount of increase in the expression of the CYB2P polypeptide is at least about 500% compared to the level expression in the parental cells grown under equivalent conditions.

5. In some embodiments of the modified cells of any of paragraphs 1-3, the amount of increase in the production of mRNA encoding the CYB2P polypeptide is at least about 1,000% compared to the level in the parental cells grown under equivalent conditions.

6. In some embodiments of the modified cells of any of paragraphs 1-3, the amount of increase in the production of mRNA encoding the CYB2P polypeptide is at least about 5,000% compared to the level in the parental cells grown under equivalent conditions.

7. In some embodiments of the modified cells of any of paragraphs 1-6, the cells further comprise an exogenous gene encoding a carbohydrate processing enzyme.

8. In some embodiments, the modified cells of any of paragraphs 1-7, further comprise a PKL pathway.

9. In some embodiments, the modified cells of any of paragraphs 1-8, further comprise an alteration in the glycerol pathway and/or the acetyl-CoA pathway.

10. In some embodiments, the modified cells of any of paragraphs 1-9, further comprise an alternative pathway for making ethanol.

11. In some embodiments of the modified cells of any of paragraphs 1-10, the cells are of a Saccharomyces spp.

12. In another aspect, a method for increased production of alcohol from yeast cells grown on a carbohydrate substrate is provided, comprising: introducing into parental yeast cells a genetic alteration that increases the production of CYB2P polypeptides compared to the amount produced in the parental cells.

13. In some embodiments of the method of paragraph 12, the cells having the introduced genetic alteration are the modified cells are the cells of any of paragraphs 1-3 and 7-11.

14. In some embodiments of the method of paragraph 12 or 13, the increased production of alcohol is at least 0.2%, at least 0.5%, at least 0.7% or at least 1.0%.

15. In some embodiments of the method of any of paragraphs 12-14, CYB2P polypeptides are over-expressed by at least 5-fold, at least 10-fold, at least 50-fold, at least 80-fold, or even at least 100-fold.

These and other aspects and embodiments of present modified cells and methods will be apparent from the description, including any accompanying Drawings/Figures.

### DETAILED DESCRIPTION

### I. Definitions

Prior to describing the present yeast and methods in detail, the following terms are defined for clarity. Terms not defined should be accorded their ordinary meanings as used in the relevant art.

As used herein, the term “alcohol” refers to an organic compound in which a hydroxyl functional group (—OH) is bound to a saturated carbon atom.

As used herein, the terms “yeast cells,” “yeast strains,” or simply “yeast” refer to organisms from the phyla Ascomycota and Basidiomycota. Exemplary yeast is budding yeast from the order Saccharomycetales. Particular examples of yeast are Saccharomyces spp., including but not limited to S. cerevisiae. Yeast include organisms used for the production of fuel alcohol as well as organisms used for the production of potable alcohol, including specialty and proprietary yeast strains used to make distinctive-tasting beers, wines, and other fermented beverages.

As used herein, the phrase “engineered yeast cells,” “variant yeast cells,” “modified yeast cells,” or similar phrases, refer to yeast that include genetic modifications and characteristics described herein. Variant/modified yeast do not include naturally occurring yeast.

As used herein, the terms “polypeptide” and “protein” (and their respective plural forms) are used interchangeably to refer to polymers of any length comprising amino acid residues linked by peptide bonds. The conventional one-letter or three-letter codes for amino acid residues are used herein and all sequence are presented from an N-terminal to C-terminal direction. The polymer can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, functionally and/or structurally similar proteins are considered to be “related proteins,” or “homologs.” Such proteins can be derived from organisms of different genera and/or species, or different classes of organisms (e.g., bacteria and fungi), or artificially designed. Related proteins also encompass homologs determined by primary sequence analysis, determined by secondary or tertiary structure analysis, or determined by immunological cross-reactivity, or determined by their functions.

As used herein, the term “homologous protein” refers to a protein that has similar activity and/or structure to a reference protein. It is not intended that homologs necessarily be evolutionarily related. Thus, it is intended that the term encompass the same, similar, or corresponding enzyme(s) (i.e., in terms of structure and function) obtained from different organisms. In some embodiments, it is desirable to identify a homolog that has a quaternary, tertiary and/or primary structure similar to the reference protein. In some embodiments, homologous proteins induce similar immunological response(s) as a reference protein. In some embodiments, homologous proteins are engineered to produce enzymes with desired activity(ies).

The degree of homology between sequences can be determined using any suitable method known in the art (see, e.g., Smith and Waterman (1981) Adv. Appl. Math. 2:482; Needleman and Wunsch (1970) J. Mol. Biol., 48:443; Pearson and Lipman (1988) Proc. Natl. Acad. Sci. USA 85:2444; programs such as GAP, BESTFIT, FASTA, and TFASTA in the Wisconsin Genetics Software Package (Genetics Computer Group, Madison, WI); and Devereux et al. (1984) Nucleic Acids Res. 12:387-95).

For example, PILEUP is a useful program to determine sequence homology levels. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle, (Feng and Doolittle (1987) J. Mol. Evol. 35:351-60). The method is similar to that described by Higgins and Sharp ((1989) CABIOS 5:151-53). Useful PILEUP parameters including a default gap weight of 3.00, a default gap length weight of 0.10, and weighted end gaps. Another example of a useful algorithm is the BLAST algorithm, described by Altschul et al. ((1990) J. Mol. Biol. 215:403-10) and Karlin et al. ((1993) Proc. Natl. Acad. Sci. USA 90:5873-87). One particularly useful BLAST program is the WU-BLAST-2 program (see, e.g., Altschul et al. (1996) Meth. Enzymol. 266:460-80). Parameters “W,” “T,” and “X” determine the sensitivity and speed of the alignment. The BLAST program uses as defaults a word-length (W) of 11, the BLOSUM62 scoring matrix (see, e.g., Henikoff and Henikoff (1989) Proc. Natl. Acad. Sci. USA 89:10915) alignments (B) of 50, expectation (E) of 10, M′5, N′-4, and a comparison of both strands.

As used herein, the phrases “substantially similar” and “substantially identical,” in the context of at least two nucleic acids or polypeptides, typically means that a polynucleotide or polypeptide comprises a sequence that has at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Percent sequence identity is calculated using CLUSTAL W algorithm with default parameters. See Thompson et al. (1994) Nucleic Acids Res. 22:4673-4680. Default parameters for the CLUSTAL W algorithm are:

| | |
|---|---|
| Gap opening penalty: | 10.0 |
| Gap extension penalty: | 0.05 |
| Protein weight matrix: | BLOSUM series |
| DNA weight matrix: | IUB |
| Delay divergent sequences %: | 40 |
| Gap separation distance: | 8 |
| DNA transitions weight: | 0.50 |
| List hydrophilic residues: | GPSNDQEKR |
| Use negative matrix: | OFF |
| Toggle Residue specific penalties: | ON |
| Toggle hydrophilic penalties: | ON |
| Toggle end gap separation penalty | OFF |

Another indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, the term “gene” is synonymous with the term “allele” in referring to a nucleic acid that encodes and directs the expression of a protein or RNA. Vegetative forms of filamentous fungi are generally haploid, therefore a single copy of a specified gene (i.e., a single allele) is sufficient to confer a specified phenotype. The term “allele” is generally preferred when an organism contains more than one similar genes, in which case each different similar gene is referred to as a distinct “allele.”

As used herein, “constitutive” expression refers to the production of a polypeptide encoded by a particular gene under essentially all typical growth conditions, as opposed to “conditional” expression, which requires the presence of a particular substrate, temperature, or the like to induce or activate expression.

As used herein, the term “expressing a polypeptide” and similar terms refers to the cellular process of producing a polypeptide using the translation machinery (e.g., ribosomes) of the cell.

As used herein, “over-expressing a polypeptide,” “increasing the expression of a polypeptide,” and similar terms, refer to expressing a polypeptide at higher-than-normal levels compared to those observed with parental or “wild-type cells that do not include a specified genetic modification.

As used herein, an “expression cassette” refers to a DNA fragment that includes a promoter, and amino acid coding region and a terminator (i.e., promoter::amino acid coding region::terminator) and other nucleic acid sequence needed to allow the encoded polypeptide to be produced in a cell. Expression cassettes can be exogenous (i.e., introduced into a cell) or endogenous (i.e., extant in a cell).

As used herein, the terms “fused” and “fusion” with respect to two DNA fragments, such as a promoter and the coding region of a polypeptide refer to a physical linkage causing the two DNA fragments to become a single molecule.

As used herein, the terms “wild-type” and “native” are used interchangeably and refer to genes, proteins or strains found in nature, or that are not intentionally modified for the advantage of the presently described yeast.

As used herein, the term “protein of interest” refers to a polypeptide that is desired to be expressed in modified yeast. Such a protein can be an enzyme, a substrate-binding protein, a surface-active protein, a structural protein, a selectable marker, a signal transducer, a receptor, a transporter, a transcription factor, a translation factor, a co-factor, or the like, and can be expressed. The protein of interest is encoded by an endogenous gene or a heterologous gene (i.e., gene of interest”) relative to the parental strain. The protein of interest can be expressed intracellularly or as a secreted protein.

As used herein, “disruption of a gene” refers broadly to any genetic or chemical manipulation, i.e., mutation, that substantially prevents a cell from producing a function gene product, e.g., a protein, in a host cell. Exemplary methods of disruption include complete or partial deletion of any portion of a gene, including a polypeptide-coding sequence, a promoter, an enhancer, or another regulatory element, or mutagenesis of the same, where mutagenesis encompasses substitutions, insertions, deletions, inversions, and combinations and variations, thereof, any of which mutations substantially prevent the production of a function gene product. A gene can also be disrupted using CRISPR, RNAi, antisense, or any other method that abolishes gene expression. A gene can be disrupted by deletion or genetic manipulation of non-adjacent control elements. As used herein, “deletion of a gene,” refers to its removal from the genome of a host cell. Where a gene includes control elements (e.g., enhancer elements) that are not located immediately adjacent to the coding sequence of a gene, deletion of a gene refers to the deletion of the coding sequence, and optionally adjacent enhancer elements, including but not limited to, for example, promoter and/or terminator sequences, but does not require the deletion of non-adjacent control elements. Deletion of a gene also refers to the deletion a part of the coding sequence, or a part of promoter immediately or not immediately adjacent to the coding sequence, where there is no functional activity of the interested gene existed in the engineered cell.

As used herein, the terms “genetic manipulation” and “genetic alteration” are used interchangeably and refer to the alteration/change of a nucleic acid sequence. The alteration can include but is not limited to a substitution, deletion, insertion or chemical modification of at least one nucleic acid in the nucleic acid sequence.

As used herein, a “functional polypeptide/protein” is a protein that possesses an activity, such as an enzymatic activity, a binding activity, a surface-active property, a signal transducer, a receptor, a transporter, a transcription factor, a translation factor, a co-factor, or the like, and which has not been mutagenized, truncated, or otherwise modified to abolish or reduce that activity. Functional polypeptides can be thermostable or thermolabile, as specified.

As used herein, “a functional gene” is a gene capable of being used by cellular components to produce an active gene product, typically a protein. Functional genes are the antithesis of disrupted genes, which are modified such that they cannot be used by cellular components to produce an active gene product, or have a reduced ability to be used by cellular components to produce an active gene product.

As used herein, yeast cells have been “modified to prevent the production of a specified protein” if they have been genetically or chemically altered to prevent the production of a functional protein/polypeptide that exhibits an activity characteristic of the wild-type protein. Such modifications include, but are not limited to, deletion or disruption of the gene encoding the protein (as described, herein), modification of the gene such that the encoded polypeptide lacks the aforementioned activity, modification of the gene to affect post-translational processing or stability, and combinations, thereof.

As used herein, “attenuation of a pathway” or “attenuation of the flux through a pathway,” i.e., a biochemical pathway, refers broadly to any genetic or chemical manipulation that reduces or completely stops the flux of biochemical substrates or intermediates through a metabolic pathway. Attenuation of a pathway may be achieved by a variety of well-known methods. Such methods include but are not limited to: complete or partial deletion of one or more genes, replacing wild-type alleles of these genes with mutant forms encoding enzymes with reduced catalytic activity or increased Km values, modifying the promoters or other regulatory elements that control the expression of one or more genes, engineering the enzymes or the mRNA encoding these enzymes for a decreased stability, misdirecting enzymes to cellular compartments where they are less likely to interact with substrate and intermediates, the use of interfering RNA, and the like.

As used herein, “aerobic fermentation” refers to growth in the presence of oxygen.

As used herein, “anaerobic fermentation” refers to growth in the absence of oxygen.

As used herein, the expression “end of fermentation” refers to the stage of fermentation when the economic advantage of continuing fermentation to produce a small amount of additional alcohol is exceeded by the cost of continuing fermentation in terms of fixed and variable costs. In a more general sense, “end of fermentation” refers to the point where a fermentation will no longer produce a significant amount of additional alcohol, i.e., no more than about 1% additional alcohol, or no more substrate left for further alcohol production.

As used herein, the expression “carbon flux” refers to the rate of turnover of carbon molecules through a metabolic pathway. Carbon flux is regulated by enzymes involved in metabolic pathways, such as the pathway for glucose metabolism and the pathway for maltose metabolism.

As used herein, the singular articles “a,” “an” and “the” encompass the plural referents unless the context clearly dictates otherwise. All references cited herein are hereby incorporated by reference in their entirety. The following abbreviations/acronyms have the following meanings unless otherwise specified:
° C. degrees Centigrade
AA α-amylase
bp base pairs
CYB2 cytochrome B2
DNA deoxyribonucleic acid
ds or DS dry solids
EC enzyme commission
EtOH ethanol
FG FERMAX™ Gold
g or gm gram
g/L grams per liter
GA glucoamylase
H₂O water
HPLC high performance liquid chromatography
hr or h hour
kg kilogram
M molar
mg milligram
min minute
mL or ml milliliter
mM millimolar
N normal
nm nanometer
PCR polymerase chain reaction
ppm parts per million
STL1 sugar transporter-like polypeptide
Δ relating to a deletion
μg microgram
μL and μl microliter
μM micromolar

### II. Modified Yeast Cells Having Increased Cyb2p Expression

In Saccharomyces cerevisiae, cytochrome B2 (CYB2; L-lactate dehydrogenase) is a component of the mitochondrial intermembrane space. It converts L-lactate to pyruvate as it converts NAD+ to NADH along with the reverse reaction. CYB2 is required for lactate utilization. Its expression is induced by lactate and repressed by glucose and anaerobic conditions. The present compositions and methods are based on the discovery that overexpression of CYB2 at the proper levels can increase ethanol production in Saccharomyces.

In some embodiments, the increase in the amount of CYB2P polypeptides produced by the modified cells is an increase of at least 200%, at least 500%, at least 1000%, at least 2,500%, or even at least 5,000%, or more, during fermentation compared to the amount of CYB2P polypeptides produced by parental cells grown under the same conditions.

In some embodiments, the increase in the amount of CYB2P polypeptides produced by the modified cells is a at least 2-fold, at least, 5-fold, at least 10-fold, at least 25-fold, or even at least 50-fold, or more, during fermentation compared to the amount of CYB2P polypeptides produced by parental cells grown under the same conditions.

In some embodiments, the increase in the strength of the promoter used to control expression of the CYB2P polypeptides produced by the modified cells is at least 2-fold, at least, 5-fold, at least 10-fold, at least 25-fold, at least 50-fold, at least 70-fold, at least 100-fold, or more, during fermentation compared to strength of the native promoter controlling CYB2P expression.

In some embodiments, the increase in ethanol production by the modified cells is an increase of at least 0.2%, at least 0.5%, at least 0.75%, at least 0.9%, at least 1.0% or more, compared to the amount of ethanol produced by parental cells grown under the same conditions.

Preferably, increased Cyb2p expression is achieved by genetic manipulation using sequence-specific molecular biology techniques, as opposed to chemical mutagenesis, which is generally not targeted to specific nucleic acid sequences. However, chemical mutagenesis is not excluded as a method for making modified yeast cells.

In some embodiments, the present compositions and methods involve introducing into yeast cells a nucleic acid capable of directing the over-expression, or increased expression, of a Cyb2p polypeptide. Particular methods include but are not limited to (i) introducing an exogenous expression cassette for producing the polypeptide into a host cell, optionally in addition to an endogenous expression cassette, (ii) substituting an exogenous expression cassette with an endogenous cassette that allows the production of an increased amount of the polypeptide, (iii) modifying the promoter of an endogenous expression cassette to increase expression, (iv) increase copy number of the same or different cassettes for over-expression of CYB2, and/or (v) modifying any aspect of the host cell to increase the half-life of the polypeptide in the host cell.

In some embodiments, the parental cell that is modified already includes a gene of interest, such as a gene encoding a selectable marker, carbohydrate-processing enzyme, or other polypeptide. In some embodiments, a gene of introduced is subsequently introduced into the modified cells.

In some embodiments, the parental cell that is modified already includes an engineered pathway of interest, such as a PKL pathway to increases ethanol production, or any other pathway to increase alcohol production.

The amino acid sequence of the exemplified S. cerevisiae Cyb2p polypeptide is shown, below, as SEQ ID NO: 1:

MLKYKPLLKI SKNCEAAILR ASKTRINTIR AYGSTVPKSK
SFEQDSRKRT QSWTALRVGA ILAATSSVAY LNWHNGQIDN
EPKLDMNKQK ISPAEVAKHN KPDDCWVVIN GYVYDLTREL
PNHPGGQDVI KENAGKDVTA IFEPLHAPNV IDKYIAPEKK
LGPLQGSMPP ELVCPPYAPG ETKEDIARKE QLKSLLPPLD
NIINLYDFEY LASQTLTKQA WAYYSSGAND EVTHRENHNA
YHRIFFKPKI LVDVRKVDIS TDMLGSHVDV PEYVSATALC
KLGNPLEGEK DVARGCGQGV TKVPQMISTL ASCSPEEIIE
AAPSDKQIQW YQLYVNSDRK ITDDLVKNVE KLGVKALEVT
VDAPSLGQRE KDMKLKESNT KAGPKAMKKT NVEESQGASR
ALSKFIDPSL TWKDIEELKK KTKLPIVIKG VQRTEDVIKA
AEIGVSGVVL SNHGGRQLDF SRAPIEVLAE TMPILEQRNL
KDKLEVEVDG GVRRGTDVLK ALCLGAKGVG LGRPFLYANS
CYGRNGVEKA IEILRDEIEM SMRLLGVTSI AELKPDLLDL
STLKARTVGV PNDVLYNEVY EGPTLTEFED A*

In some embodiments of the present compositions and methods, the amino acid sequence of the Cyb2p polypeptide that is over-expressed in modified yeast cells has at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99% identity, to SEQ ID NO: 1.

Amino acid sequence searching identified several known Cyb2p molecules within 90% amino acid sequence identity of the exemplified molecule (i.e., SEQ ID NO: 1) with similar annotations. In particular embodiments of the present compositions and methods, the amino acid sequence of the Cyb2p polypeptide that is over-expressed in modified yeast cells has at least 90%, at least 91%, at least 92%, at least 93%, at least 94%, at least 95%, at least 96%, at least 97%, at least 98%, or even at least 99% identity, to SEQ ID NO: 1 and/or one or more of the Cyb2p amino acid sequences referred to in Table 1.

TABLE 1
Cytochrome B2 proteins from public databases

| | | % identity | |
|---|---|---|---|
| | | with | GenBank |
| | | exemplified | Accession |
| Gene Name | Source organism | Cyb2p | #s |
| Cyb2p | Saccharomyces cerevisiae | 100.00% | NP_013658.1 |
| | S288C | | |
| Cyb2p | Saccharomyces cerevisiae | 99.83% | AJS85393.1 |
| | YJM1342 | | |
| Cytochrome | Saccharomyces cerevisiae | 99.83% | ONH72646.1 |
| b2, | | | |
| mitochondrial | | | |
| Cyb2p | Saccharomyces cerevisiae | 99.83% | AJS67503.1 |
| | YJM456 | | |
| Cyb2p | Saccharomyces cerevisiae | 99.83% | AJS66626.1 |
| | YJM451 | | |
| Cyb2p | Saccharomyces cerevisiae | 99.83% | AJS62699.1 |
| | YJM195 | | |
| Cyb2p | Saccharomyces cerevisiae | 99.83% | AJP40658.1 |
| | YJM1078 | | |
| Cyb2p | Saccharomyces cerevisiae | 99.83% | AJS89762.1 |
| | YJM1399 | | |
| Cyb2p | Saccharomyces cerevisiae | 99.66% | AJS69692.1 |
| | YJM627 | | |
| Cyb2p | Saccharomyces cerevisiae x | 91.54% | EHN00948.1 |
| | Saccharomyces kudriavzevii | | |
| | VIN7 | | |

### III. Modified Yeast Cells Having Increased Cyb2p Expression in Combination with Genes of an Exogenous PKL Pathway

Increased expression of Cyb2p can be combined with expression of genes in the PKL pathway to further increase ethanol production. Engineered yeast cells having a heterologous PKL pathway have been previously described in WO2015148272 (Miasnikov et al.). These cells express heterologous phosphoketolase (PKL), phosphotransacetylase (PTA) and acetylating acetyl dehydrogenase (AADH), optionally with other enzymes, to channel carbon flux away from the glycerol pathway and toward the synthesis of acetyl-CoA, which is then converted to ethanol. Such modified cells are capable of increased ethanol production in a fermentation process when compared to otherwise-identical parent yeast cells.

### IV. Combination of Increased Cyb2p Production with Other Mutations that Affect Alcohol Production

In some embodiments, in addition to expressing increased amounts of Cyb2p polypeptides, optionally in combination with introducing an exogenous PKL pathway, the present modified yeast cells include additional modifications that affect ethanol production.

The modified cells may further include mutations that result in attenuation of the native glycerol biosynthesis pathway and/or reuse glycerol pathway, which are known to increase alcohol production. Methods for attenuation of the glycerol biosynthesis pathway in yeast are known and include reduction or elimination of endogenous NAD-dependent glycerol 3-phosphate dehydrogenase (GPD) or glycerol phosphate phosphatase activity (GPP), for example by disruption of one or more of the genes GPD1, GPD2, GPP1 and/or GPP2. See, e.g., U.S. Pat. No. 9,175,270 (Elke et al.), U.S. Pat. No. 8,795,998 (Pronk et al.) and U.S. Pat. No. 8,956,851 (Argyros et al.). Methods to enhance the reuse glycerol pathway by over expression of glycerol dehydrogenase (GCY1) and dihydroxyacetone kinase (DAK1) to convert glycerol to dihydroxyacetone phosphate (Zhang et al. (2013) J. Ind. Microbiol. Biotechnol. 40:1153-60).

The modified yeast may further feature increased acetyl-CoA synthase (also referred to acetyl-CoA ligase) activity (EC 6.2.1.1) to scavenge (i.e., capture) acetate produced by chemical or enzymatic hydrolysis of acetyl-phosphate (or present in the culture medium of the yeast for any other reason) and converts it to Ac-CoA. This partially reduces the undesirable effect of acetate on the growth of yeast cells and may further contribute to an improvement in alcohol yield. Increasing acetyl-CoA synthase activity may be accomplished by introducing a heterologous acetyl-CoA synthase gene into cells, increasing the expression of an endogenous acetyl-CoA synthase gene and the like.

In some embodiments the modified cells may further include a heterologous gene encoding a protein with NAD⁺-dependent acetylating acetaldehyde dehydrogenase activity and/or a heterologous gene encoding a pyruvate-formate lyase. The introduction of such genes in combination with attenuation of the glycerol pathway is described, e.g., in U.S. Pat. No. 8,795,998 (Pronk et al.). In some embodiments of the present compositions and methods the yeast expressly lacks a heterologous gene(s) encoding an acetylating acetaldehyde dehydrogenase, a pyruvate-formate lyase or both.

In some embodiments, the present modified yeast cells may further over-express a sugar transporter-like (STL1) polypeptide to increase the uptake of glycerol (see, e.g., Ferreira et al. (2005) Mol. Biol. Cell. 16:2068-76; Dušková et al. (2015) Mol. Microbiol. 97:541-59 and WO 2015023989 A1) to increase ethanol production and reduce acetate.

In some embodiments, the present modified yeast cells further include a butanol biosynthetic pathway. In some embodiments, the butanol biosynthetic pathway is an isobutanol biosynthetic pathway. In some embodiments, the isobutanol biosynthetic pathway comprises a polynucleotide encoding a polypeptide that catalyzes a substrate to product conversion selected from the group consisting of: (a) pyruvate to acetolactate; (b) acetolactate to 2,3-dihydroxyisovalerate; (c) 2,3-dihydroxyisovalerate to 2-ketoisovalerate; (d) 2-ketoisovalerate to isobutyraldehyde; and (e) isobutyraldehyde to isobutanol. In some embodiments, the isobutanol biosynthetic pathway comprises polynucleotides encoding polypeptides having acetolactate synthase, keto acid reductoisomerase, dihydroxy acid dehydratase, ketoisovalerate decarboxylase, and alcohol dehydrogenase activity.

In some embodiments, the modified yeast cells comprising a butanol biosynthetic pathway further comprise a modification in a polynucleotide encoding a polypeptide having pyruvate decarboxylase activity. In some embodiments, the yeast cells comprise a deletion, mutation, and/or substitution in an endogenous polynucleotide encoding a polypeptide having pyruvate decarboxylase activity. In some embodiments, the polypeptide having pyruvate decarboxylase activity is selected from the group consisting of: PDC1, PDC5, PDC6, and combinations thereof. In some embodiments, the yeast cells further comprise a deletion, mutation, and/or substitution in one or more endogenous polynucleotides encoding FRA2, ALD6, ADH1, GPD2, BDH1, and YMR226C.

### V. Combination of Increased Expression Cyb2p with Other Beneficial Mutations

In some embodiments, in addition to increased expression of Cyb2p polypeptides, optionally in combination with other genetic modifications provide a benefit, the present modified yeast cells further include any number of additional genes of interest encoding proteins of interest. Additional genes of interest may be introduced before, during, or after genetic manipulations that result in the increased production of Cyb2p polypeptides. Proteins of interest, include selectable markers, carbohydrate-processing enzymes, and other commercially-relevant polypeptides, including but not limited to an enzyme selected from the group consisting of a dehydrogenase, a transketolase, a phosphoketolase, a transladolase, an epimerase, a phytase, a xylanase, a β-glucanase, a phosphatase, a protease, an α-amylase, a β-amylase, a glucoamylase, a pullulanase, an isoamylase, a cellulase, a trehalase, a lipase, a pectinase, a polyesterase, a cutinase, an oxidase, a transferase, a reductase, a hemicellulase, a mannanase, an esterase, an isomerase, a pectinases, a lactase, a peroxidase and a laccase. Proteins of interest may be secreted, glycosylated, and otherwise-modified.

### VI. Use of the Modified Yeast for Increased Alcohol Production

The present compositions and methods include methods for increasing alcohol production and/or reducing glycerol production, in fermentation reactions. Such methods are not limited to a particular fermentation process. The present engineered yeast is expected to be a “drop-in” replacement for convention yeast in any alcohol fermentation facility. While primarily intended for fuel alcohol production, the present yeast can also be used for the production of potable alcohol, including wine and beer.

### VII. Yeast Cells Suitable for Modification

Yeasts are unicellular eukaryotic microorganisms classified as members of the fungus kingdom and include organisms from the phyla Ascomycota and Basidiomycota. Yeast that can be used for alcohol production include, but are not limited to, Saccharomyces spp., including S. cerevisae, as well as Kluyveromyces, Lachancea and Schizosaccharomyces spp. Numerous yeast strains are commercially available, many of which have been selected or genetically engineered for desired characteristics, such as high alcohol production, rapid growth rate, and the like. Some yeasts have been genetically engineered to produce heterologous enzymes, such as glucoamylase or α-amylase.

### VIII. Substrates and Products

Alcohol production from a number of carbohydrate substrates, including but not limited to corn starch, sugar cane, cassava, and molasses, is well known, as are innumerable variations and improvements to enzymatic and chemical conditions and mechanical processes. The present compositions and methods are believed to be fully compatible with such substrates and conditions.

Alcohol fermentation products include organic compound having a hydroxyl functional group (—OH) is bound to a carbon atom. Exemplary alcohols include but are not limited to methanol, ethanol, n-propanol, isopropanol, n-butanol, isobutanol, n-pentanol, 2-pentanol, isopentanol, and higher alcohols. The most commonly made fuel alcohols are ethanol, and butanol.

These and other aspects and embodiments of the present yeast strains and methods will be apparent to the skilled person in view of the present description. The following examples are intended to further illustrate, but not limit, the compositions and methods.

### EXAMPLES

### Example 1

### Materials and Methods

### Liquefact Preparation:

Liquefact (corn mash slurry) was prepared by adding 600 ppm of urea, 0.124 SAPU/g ds acid fungal protease, 0.33 GAU/g ds variant Trichoderma reesei glucoamylase and 1.46 SSCU/g ds Aspergillus kawachii α-amylase, adjusted to a pH of 4.8 with sulfuric acid.

### AnKom Assays:

300 μL of concentrated yeast overnight culture was added to each of a number ANKOM bottles filled with 50 g prepared liquefact (see above) to a final OD of 0.3. The bottles were then incubated at 32° C. with shaking at 150 RPM for 55 hours.

### HPLC Analysis:

Samples of the cultures from AnKom assays were collected in Eppendorf tubes by centrifugation for 12 minutes at 14,000 RPM. The supernatants were filtered using 0.2 μM PTFE filters and then used for HPLC (Agilent Technologies 1200 series) analysis with the following conditions: Bio-Rad Aminex HPX-87H columns, running at a temperature of 55° C. with a 0.6 ml/min isocratic flow in 0.01 N H₂SO₄ and a 2.5 μl injection volume. Calibration standards were used for quantification of the of acetate, ethanol, glycerol, glucose and other molecules. Unless otherwise indicated, all values are reported in g/L.

### RNA-Seq Analysis:

RNA was prepared from individual samples according to the TRIzol method (Life-Tech, Rockville, MD). The RNA was then cleaned up with Qiagen RNeasy Mini Kit (Qiagen, Germantown, MD). The cDNA from total mRNA in individual samples was generated using Applied Biosystems High Capacity cDNA Reverse Transcription kit (Thermo Fisher Scientific, Wilmington, Delaware). The prepared cDNA of each sample was sequenced using the shotgun method, and then quantified with respect to individual genes. The results are reported as reads per kilobase ten million transcripts (RPK10M), and used to quantify the amount of each transcript in a sample.

### Example 2

### Expression of CYB2 in Yeast

RNA-Seq analysis was performed as described in Example 1. As summarized in Table 2, yeast over-expressing STL1 express CYB2 at significantly higher levels than parental yeast, i.e., FERMAX™ Gold (Martrex Inc., Minnesota, USA; herein abbreviated “FG”), with or without an exogenous phosphoketolase (PKL) pathway. Expression levels are expressed as reads per kilobase ten million transcripts (RPK10M).

TABLE 2
RNA-Seq analysis of CYB2 expression if different strains

| | Strain | | |
|---|---|---|---|
| Time | FG | FG + PKL | FG + PKL + STL1 |
| 24 hr | 782 | 724 | 1012 |
| 48 hr | 8981 | 6481 | 16155 |

The results suggested that the gene may be a target for increasing ethanol production in engineered yeast.

### Example 3

### Promoter Selection for Over Expression of CYB2

RNA-Seq analysis was performed to identify a promoter for over-expression of CYB2 during fermentation. Analysis was performed as described in Example 1. Consistent with the results described in Example 2, CYB2 expression in FG was low in the first 24 hr of fermentation. In contrast, the actin (ACT1) gene was highly expressed at 6, 15 and 24 hr into fermentation. Accordingly, the ACT1 promoter was selected for overexpressing CYB2 in yeast.

TABLE 3
Transcription profiles of CYB2 and ACT1 in FG
during fermentation

| | Time (hr) | | |
|---|---|---|---|
| Strain | 6 | 15 | 24 |
| ACT1 | 14,906 | 26,381 | 30,898 |
| CYB2 | 2203 | 317 | 920 |

### Example 4

### Preparation of a Cyb2p Expression Cassette

The CYB2 gene (YML054C locus. SEQ ID: 2) of Saccharomyces cerevisiae was synthesized to generate CYB2s. The ACT1 promoter (YFL039C locus; SEQ ID NO: 3) and FBA1 terminator (YKL060C locus; SEQ ID NO: 4) were operably linked to the coding sequence to generate the ACT1Pro::CYB2ss::Fba1Ter expression cassette. This expression cassette was introduced at position 350000 of Chromosome II of FERMAX™ Gold (Martrex Inc., Minnesota, USA; herein abbreviated, “FG”), a well-known fermentation yeast used in the grain ethanol industry. The expected insertion of the CYB2s expression cassette in the two parental strains was confirmed by PCR.

The amino acid sequence of the CYB2 polypeptide
is shown, below, as SEQ ID NO: 1:
MLKYKPLIKI SKNCEAAILR ASKTRLNTIR AYGSTVPKSK
SFEQDSRKRT QSWTALRVGA ILAATSSVAY LNWHNGQIDN
EPKLDMNKQK ISPAEVAKHN KPDDCWVVIN GYVYDLTREL
PNHPGGQDVI KFNAGKDVTA IFEPLHAPNV IDKYIAPEKK
LGPLQGSMPP ELVCPPYAPG ETKEDIARKE QLKSLLPPLD
NIINLYDFEY LASQTLTKQA WAYYSSGAND EVTHRENHNA
YHRIFFKPKI LVDVRKVDIS TDMLGSHVDV PFYVSATALC
KLGNPLEGEK DVARGCGQGV TKVPQMISTI ASCSPEEIIE
AAPSDKQIQW YQLYVNSDRK ITDDLVKNVE KLGVKALFVT
VDAPSLGQRE KDMKLKFSNT KAGPKAMKKT NVEESQGASR
ALSKFIDPSL TWKDIEELKK KTKLPIVIKG VQRTEDVIKA
AEIGVSGVVL SNHGGRQLDE SRAPIEVLAE TMPILEQRNL
KDKLEVEVDG GVRRGTDVLK ALCLGAKGVG LGRPFLYANS
CYGRNGVEKA IEILRDEIEM SMRLLGVTSI AELKPDLLDL
STLKARTVGV PNDVLYNEVY EGPTLTEFED A*
The DNA sequence of CYB2-coding region is
shown, below, as SEQ ID NO: 2:
ATGTTGAAGTACAAGCCATTGCTAAAGATTTCCAAGAACTGTGAA
GCTGCCATCTTGAGAGCTTCCAAGACCAGATTGAACACTATCAGA
GCTTACGGTTCTACCGTTCCAAAGTCCAAGTCTTTCGAACAAGAC
TCCAGAAAGCGTACACAATCTTGGACTGCCTTGAGAGTCGGTGCT
ATTCTAGCTGCCACCTCTTCCGTTGCTTACTTGAACTGGCACAAT
GGTCAAATCGACAACGAACCAAAGTTGGATATGAACAAGCAAAAG
ATTTCTCCAGCTGAAGTTGCCAAGCACAACAAACCAGACGATTGT
TGGGTCGTTATCAACGGTTACGTCTACGACTTAACCAGATTTCTA
CCCAATCATCCAGGTGGCCAAGACGTTATCAAGTTCAACGCTGGC
AAGGATGTCACTGCTATCTTCGAACCATTGCACGCTCCAAACGTC
ATCGACAAGTACATTGCTCCAGAAAAGAAATTGGGTCCATTGCAA
GGTTCTATGCCTCCAGAATTGGTCTGTCCTCCATACGCTCCAGGT
GAAACCAAGGAAGACATTGCCAGAAAGGAACAACTAAAGTCCTTG
TTACCACCTTTGGACAACATTATCAACTTGTACGATTTCGAATAC
TTGGCTTCTCAAACTTTGACCAAGCAAGCCTGGGCTTACTACAGC
TCTGGTGCCAACGACGAAGTCACTCACAGAGAAAACCACAATGCT
TACCACAGAATCTTTTTCAAGCCAAAGATTTTGGTTGACGTCAGA
AAGGTTGACATCTCTACCGATATGTTGGGTTCTCACGTTGACGTG
CCATTTTACGTCTCTGCTACTGCCTTGTGCAAGTTGGGAAATCCA
TTGGAAGGTGAGAAGGACGTTGCCAGAGGTTGTGGCCAAGGTGTC
ACCAAGGTTCCACAAATGATCTCCACTTTAGCTTCATGTTCTCCA
GAGGAAATTATCGAAGCTGCTCCATCCGACAAGCAAATCCAATGG
TACCAATTGTACGTCAACTCCGACAGAAAGATTACCGACGATTTG
GTCAAGAACGTCGAAAAGTTGGGTGTCAAGGCCTTATTTGTTACT
GTCGATGCTCCAAGCCTAGGTCAAAGAGAAAAGGACATGAAGTTG
AAGTTTTCCAACACAAAGGCTGGTCCCAAGGCTATGAAGAAAACC
AATGTTGAAGAGTCICAAGGTGCCTCCAGAGCTTTGTCCAAGTTC
ATCGACCCATCITTAACTTGGAAGGATATCGAAGAGTTGAAGAAA
AAGACCAAGCTACCAATTGTTATCAAGGGTGTTCAAAGAACCGAA
GACGTTATCAAGGCTGCCGAAATCGGTGTCTCTGGTGTTGTCCTA
TCCAACCACGGTGGCAGACAATTGGATTTTTCCCGTGCTCCAATC
GAAGTCTTGGCTGAAACCATGCCAATCTTGGAACAAAGAAACTTG
AAGGACAAGTTGGAAGTTTTTGTCGATGGTGGCGTCAGACGTGGT
ACAGACGTCTTGAAGGCTCTATGTTTGGGTGCCAAGGGTGTTGGC
TTAGGTAGACCATTCTTGTACGCCAACTCCTGTTACGGCAGAAAC
GGTGTCGAAAAGGCTATCGAAATCTTGAGAGACGAAATTGAAATG
TCTATGCGTCTATTGGGTGTTACTTCCATCGCTGAATTGAAGCCA
GATTTGCTAGACTTATCTACTTTGAAGGCCAGAACCGTIGGTGTA
CCCAACGACGTCTTGTACAACGAAGTTTACGAAGGTCCTACTTTG
ACCGAATTTGAGGATGCTTAA
The ACT1 promoter region used for CYB2s
over-expression shown, below, as SEQ ID NO: 3:
AAAGTATGTCCCAACAAAACCTGAACGAAACCACTCAGAAGAAGG
AAGACAAAGGGAACGTCAACCTGAAGGGACAGAGTTTAACCAACA
CCGGTGGGGGCTGCTGTTGAACAAGCGCGCCTCTACCTTGCAGAC
CCATATAATATAATAACTAAATAAGTAAATAAGACACACGCGAGA
ACATATATACACAATTACAGTAACAATAACAAGAGGACAAATACT
ACCAAAATGTGTGGGGAAGCGGGTAAGCTGCCACAGCAATTAATG
CACAACATTTAACCTACATTCTTCCTTATCGGATCCTCAAAACCC
TTAAAAACATATGCCTCACCCTAACATATTTTCCAATTAACCCTC
AATATTTCTCTGTCACCCGGCCTCTATTTTCCATTTTCTTCTTTA
CCCGCCAGGCGTTTTTTTCTTTCAAATTTTTTTCTTCCTTCTTCT
TTTTCTTCCACGTCCTCTTGCATAAATAAATAAACCGTTTTGAAA
CCAAACTCGCCTCTCTCTCTCCTTTTTGAAATATTTTTGGGTTTG
ITTGATCCTTTCCTTCCCAATCTCTCTTGTTTAATATATATTCAT
TTATATCACGCTCTCTTTTTATCTTCCTTTTTCTCCTCTCTCTTG
TATTCTTCCTTCCCCTTTCTACTCAAACCAAGAAGAAAAAGAAAA
GGTCAATCTTTGTTAAAGAATAGGATCTTCTACTACATCAGCTTT
TAGATTTTTCACGCTTACTGCTTTTTTCTTCCCAAGATCGAAAAT
TTACTGAATTAACA
The FBA1 terminator region used for CYB2s
over-expression shown, below, as
SEQ ID NO: 4:
GTTAATTCAAATTAATTGATATAGTTTTTTAATGAGTATTGAATC
TGTTTAGAAATAATGGAATATTATTTTTATTTATTTATTTATATT
ATTGGTCGGCTCTTTTCTTCTGAAGGTCAATGACAAAATGATATG
AAGGAAATAATGATTTCTAAAATTTTACAACGTAAGATATTTTTA
CAAAAGCCTAGCTCATCTTTTGTCATGCACTATTTTACTCACGCT
TGAAATTAACGGCCAGTCCACTGCGGAGTCATTTCAAAGTCATCC
TAATCGATCTATCGTTTTTGATAGCTCATTTTG

### Example 5

### Alcohol Production by Yeast Over-Expressing CYB2

One PCR positive strain over-expressing CYB2s under the control of the ACT1 promoter, and its parental strain FG, were tested in an Ankom assay containing 50 g liquefact. Fermentations were performed at 32° C. for 65 hours. Samples from the end of fermentation were analyzed by HPLC. The experiments were repeated several times and a typical result is summarized in Table 4. The data are the average of duplicate samples of each strain.

TABLE 4
HPLC results from FG and FG-CYB2 strains

| | Glucose | Glycerol | Acetate | Ethanol | Ethanol |
|---|---|---|---|---|---|
| Strain | (g/L) | (g/L) | (g/L) | (g/L) | increase (%) |
| FG | 1.06 | 16.8 | 0.99 | 148.1 | -0- |
| FG-CYB2 | 1.06 | 16.9 | 1.03 | 149.7 | 1.01% |

Over-expression of CYB2s resulted in about a 1.0% increase of ethanol production in FG yeast, which is recognized as a robust, high-ethanol-producing yeast for the fuel ethanol industry. These results demonstrate that CYB2 over-expression is beneficial for increasing ethanol.

1. Modified yeast cells derived from parental yeast cells, the modified cells comprising a genetic alteration that causes the modified cells to produce an increased amount of CYB2P polypeptides compared to the parental cells, wherein the modified cells produce during fermentation more ethanol compared to the amount of ethanol produced by otherwise identical parent yeast cells.

2. The modified cells of claim 1, wherein the genetic alteration comprises the introduction into the parental cells of a nucleic acid capable of directing the expression of a CYB2P polypeptide to a level above that of the parental cell grown under equivalent conditions.

3. The modified cells of claim 1, wherein the genetic alteration comprises the introduction of an expression cassette for expressing a CYB2P polypeptide.

4. The modified cells of claim 1, wherein the amount of increase in the expression of the CYB2P polypeptide is at least about 500% compared to the level expression in the parental cells grown under equivalent conditions.

5. The modified cells of claim 1, wherein the amount of increase in the production of mRNA encoding the CYB2P polypeptide is at least about 1,000% compared to the level in the parental cells grown under equivalent conditions.

6. The modified cells of claim 1, wherein the amount of increase in the production of mRNA encoding the CYB2P polypeptide is at least about 5,000% compared to the level in the parental cells grown under equivalent conditions.

7. The modified cells of claim 1, wherein the cells further comprise an exogenous gene encoding a carbohydrate processing enzyme.

8. The modified cells of claim 1, further comprising a PKL pathway.

9. The modified cells of claim 1, further comprising an alteration in the glycerol pathway and/or the acetyl-CoA pathway.

10. The modified cells of claim 1, further comprising an alternative pathway for making ethanol.

11. The modified cells of claim 1, wherein the cells are of a Saccharomyces spp.

12. A method for increased production of alcohol from yeast cells grown on a carbohydrate substrate, comprising: introducing into parental yeast cells a genetic alteration that increases the production of CYB2P polypeptides compared to the amount produced in the parental cells.

13. The method of claim 12, wherein the cells having the introduced genetic alteration are the modified cells are the cells of any of claims 1-3 and 7-11.

14. The method of claim 12, wherein the increased production of alcohol is at least 0.2%, at least 0.5%, at least 0.7% or at least 1.0%.

15. The method of claim 12, wherein CYB2P polypeptides are over-expressed by at least 5-fold, at least 10-fold, at least 50-fold, at least 80-fold, or even at least 100-fold.
